SEQUENCE LISTING (2 sequences)

SEQ ID NO: 1 - protein, 7 aa, Artificial
  Polypeptide
GHRPLDK

SEQ ID NO: 2 - protein, 8 aa, Artificial
  Polypeptide
GHRPLDKC

(19) United States
(12) Patent Application Publication
(10) Pub. No.: US 2006/0127958 A1
(43) Pub. Date: Jun. 15, 2006

(54) Immunoassay for the detection of cancer

(76) Inventors: Michael C. Cress, Santa Ana, CA (US); Ronald J. Moore, Tustin, CA (US); That T. Ngo, Irvine, CA (US)

Correspondence Address: PRESTON GATES & ELLIS LLP, 1900 MAIN STREET, SUITE 600, IRVINE, CA, 92614-7319, US

(21) Appl. No.: 11/316,295

(22) Filed: Dec. 21, 2005

Related U.S. Application Data
(63) Continuation of application No. 09/424,940, filed on Mar. 7, 2000, now abandoned.

(51) Int. Cl. G01N 33/574

(52) U.S. Cl. 435/7.23

(57) ABSTRACT

The present invention relates to a broad cancer immunoassay. Specifically, an immunoassay for peptides associated with oncogenic processes such as metastatic proteolysis is disclosed. In an illustrative embodiment, the immunoassay utilizes antibodies which bind to peptides which are generated by proteolytic processes and which contain epitopes which are masked in undegraded blood proteins such as fibrinogen. Detection of such degradation peptides in a biological sample by immunological methods allows the diagnosis of a wide variety of cancers.

### CROSS-REFERENCE TO RELATED APPLICATIONS

This application claims the benefit of U.S. Provisional Application No. 60/048,405, entitled “IMMUNOASSAY FOR THE DETECTION OF CANCER,” filed on Jun. 3, 1997, by Ngo et al., and U.S. Provisional Application No. 60/060,088 entitled “IMMUNOASSAY FOR THE DETECTION OF CANCER,” filed on Sep. 26, 1997, by Ngo et al., which are incorporated by reference herein.

### BACKGROUND OF THE INVENTION

1. Field of the Invention

This invention relates generally to immunoassays for the detection of cancer.

2. Description of Related Art

An ongoing challenge in medicine is the development of methods that permit the rapid and accurate diagnosis of disease. Despite recent advances in diagnostic technologies, current techniques for the diagnosis of many diseases are either inadequate or cost prohibitive for a wide scale application. One such illustrative disease is cancer. Many “cancer antigens” have been discovered, for example: cancer antigens CEA, CA19-9 and CA242 are used in the diagnosis and treatment of gastrointestinal cancer; cancer antigen CA125 is used in the diagnosis and treatment of ovarian cancer; cancer antigen AFP is associated with testicular and liver cancers; the CA15-3 and HER2/neu antigens are associated with breast cancers; and the PSA and PAP antigens have been shown to be associated with prostate cancer. While the identification of such antigens can be useful once a patient is identified for being at risk for specific cancer or has been diagnosed with a specific cancer, they are of limited use in identifying individuals with cancer in a general population. A general screening of the population using specific cancer antigens would be expensive due to the multiple tests required and would only detect the specific cancers for which antigens are available.

Some antigens, such as the carcinoembryonic antigen, are found in patients with a number of different cancers, such as lung, liver, pancreas, breast, head or neck, bladder, cervix and prostate, in addition to those suffering from adenocarcinoma of the colon. However, in these cases only 30% of the patients test positive. This amount is too low for this antigen to be useful as a diagnostic tool.

Cancer associated markers may arise from a variety of sources including those associated with common oncogenic processes. For example, it is known that a wide variety of tumor cells of different lineages release proteases into interstitial fluid at a higher rate than normal cells. Sylven B., “Lysosomal Enzyme Activity in the Interstitial Fluid of Solid Mouse Tumour Transplants,” Eur. J. Cancer, 4:463-474, (1968); Sylven B., “Cellular Detachment by Purified Lysosomal Cathepsin B,” Eur. J. Cancer, 4:559-562, (1968). A number of lines of evidence support the concept that this increased protease activity contributes directly to the invasiveness of tumor cells and to the destruction of the adjacent host tissue. Poole et al., “Differences in Secretion of the Proteinase Cathepsin B at the Edges of Human Breast Carcinomas and Fibroadenomas,” Nature, 273:545-547, (1978); Keppler et al., “Secretion of Cathepsin B and Tumor Invasion,” Biochem Soc. Trans., 22:43-49, (1994); Pietras et al., “Lysosomal Cathepsin B-Like Activity: Mobilization in Prereplicative and Neoplastic Epithelial Cells,” J. Histochem Cytochem, 29:440-450, (1981).

In breast cancer metastases, four classes of proteases appear to be involved in disease progression. Dickson et al., “A Novel Matrix-Degrading Protease in Hormone-Dependent Breast Cancer,” Biochem Soc. Trans., 22:49-52, (1994). These include cysteine proteases (cathepsins B and L), aspartyl proteases (cathepsin D), collagenases (metalloproteases) and serine proteases (urokinase and plasminogen). Increased expression of the collagenases has been correlated with increased invasiveness of some tumor cells. Down-regulation of these enzymes by genetic means reduces both the invasiveness and metastases of the tumor. Moreover, the addition of tissue metalloproteinase inhibitors to tumor cells blocks cell invasion in vitro. Further, the administration of either natural or synthetic metalloproteinase inhibitors has been shown to prevent metastasis in a simple lung colonization model. Goldberg et al., “Extracellular Matrix Metalloproteinases in Tumor Invasion and Metastasis,” in Regulatory Mechanisms in Breast Cancer, Lippman M E, and Dickson R B (eds), Boston, Kluwer Academic Publishers, pp. 421-440, (1990).

Protease release by tumor cells can also result in the proteolysis of plasma proteins. Theoretically the extent of proteolytic degradation of these proteins can be correlated with the activity of the tumor cells and used indirectly to evaluate their tumor burden or degree of malignancy. Therefore the identification of antigens associated with the proteolytic activity associated with malignancy should yield new markers that are associated with oncogenic processes.

There is a need in the art for the identification of antigens which are associated with universal oncogenic processes, and which are not limited to a specific type of cancer. Such pan-marker or universal marker antigen(s) will be useful for the routine screening of patients to determine if they have cancer. After an initial screening, patients with elevated concentrations of the pan-marker, when compared to a “normal” population, would be further screened to determine if they do in fact have cancer and the specific type of cancer from which they are suffering. Additionally, it is desirable that such a pan-marker is present in blood, or other biological fluids, so that testing can be performed on easily obtainable samples.

### SUMMARY OF THE INVENTION

The present invention is directed to immunoassays for the detection of cancers. In one embodiment, the invention provides a method for detecting cancer in a subject by contacting a biological sample obtained from the subject with an antibody that binds an epitope on a blood protein degradation peptide that is masked in the blood protein and determining the presence of an antibody-peptide complex. In a preferred embodiment of the invention, the blood protein is human fibrinogen and the antibody recognizes an epitope comprising the amino acids 15 to 21 of the β-chain of human fibrinogen.

A wide variety of assays for the degradation peptide may be utilized. In one embodiment, the assay is an enzyme-linked immunoadsorbent (ELISA) assay. In a preferred embodiment, the assay is a sandwich type ELISA immunoassay. Biological samples which are assayed in the present invention may be obtained from a variety of sources. In a preferred embodiment, the biological sample consists of human blood. In addition, a variety of modifications and variations of this assay are disclosed. In one embodiment, the assay includes the additional step of screening a biological sample isolated from the subject for the presence of a second tumor marker. In a preferred embodiment of this variation, the second tumor marker consists of either PSA, CEA, CA 15-3, CA 19-9 or CA 125, or a combination thereof.

A significant feature of the invention is the identification of cancer markers which comprise epitopes on endogenous proteins that are usually inaccessible to immunodetection in normal subjects. In a number of the exemplary embodiments, the invention disclosed herein offers a number of performance advantages over assays in the prior art. First, they enable immunochemical measurements of proteolytic degradation products in the presence of, and without interference by the endogenous normal protein molecules. Second, these embodiments detects multiple cancers with a high degree of specificity and sensitivity. Third, when such assays are used together with additional established organ-specific markers, the overall clinical performance is improved.

### BRIEF DESCRIPTION OF THE DRAWINGS

FIG. 1. shows a graph of the standard curve for fibrinogen digestion products (FDP) as a function of absorbance at 450 nm; and

FIG. 2. shows a graph derived from dilution of a high titer patient sample.

FIG. 3. shows a scatterplot of normalized FDP ratios of serum samples from normal subjects and patients having cancer of the breast, colon, lung, ovary or prostate.

FIG. 4. shows a scatterplot of CA 15-3 levels and FDP levels in serum samples from breast cancer patients.

FIG. 5. shows a scatterplot of CA 19-9 levels and FDP levels in serum samples from colon cancer patients.

FIG. 6. shows a scatterplot of CEA levels and FDP levels in serum samples from colon cancer patients.

FIG. 7. shows a scatterplot of CEA levels and FDP levels in serum samples from lung cancer patients.

FIG. 8. shows a scatterplot of CA 125 levels and FDP levels in serum samples from ovarian cancer patients.

FIG. 9. shows a scatterplot of PSA levels and FDP levels in serum samples from prostate cancer patients.

FIGS. 10a-d show western blots made from SDS-PAGE gels of pleural effusate from a patient with lung cancer.

FIG. 10a. is derived from a reduced gel and the probe was derived from a ring shaped particle extract.

FIG. 10b. is derived from a reduced gel and probed with a monoclonal antibody of the invention.

FIG. 10c. is derived from a non-reduced gel and the probe was derived from a ring shaped particle extract.

FIG. 10d. is derived from a non-reduced gel and probed with a monoclonal antibody of the invention.

FIGS. 11a-c show the selectivity of the assay through graphs of the standard curves for fibrinogen fragment D, fibrinogen and fibrinogen fragment E as a function of absorbance at 450 nm.

### DETAILED DESCRIPTION OF THE INVENTION

Definitions

The term “antibody” is used in the broadest sense and specifically covers monoclonal antibodies and variations thereof including antibody fragments, chimeric or other recombinant molecules that are known in the art. The term “monoclonal antibody” as used herein refers to an antibody obtained from a population of substantially homogeneous antibodies, i.e., the individual antibodies comprising the population are identical except for possible naturally-occurring mutations that may be present in minor amounts.

The term “tumor marker” as used herein is broadly defined as any one of a wide variety of peptides, nucleic acids and related molecules of which the presence or levels of are used to assess the status of oncogenic processes.

The term “masked” as used herein, for example in the context of degradation peptides, is broadly defined as peptide sequences that are not appreciably recognized or accessible by detection systems, such as antibodies, in normal endogenous proteins. A “masked” peptide may exist within the interior of a native protein, but is not exposed until the protein is degraded and an internal peptide is released or exposed.

The term “degradation peptide” as used herein is broadly defined as a peptide fragment of a larger protein which has been degraded, for example, as occurs with the proteolytic degradation of blood proteins that is observed in oncogenic processes.

The term “mammal” as used herein refers to any mammal classified as a mammal, including humans, cows, horses, dogs and cats. In a preferred embodiment of the invention, the mammal is a human.

Identification of Markers Associated with Cancer

The present invention is directed at a method of screening for cancer by detecting an epitope in a protein peptide not generally accessible on the full length protein but which becomes so upon proteolytic degradation. Such peptides are generated by the action of proteases which are involved oncogenic processes.

The present invention illustrates the association between common oncogenic processes such as proteolysis and novel cancer antigens. Proteases are associated with oncogenesis and are released at a higher rate into the interstitial fluid of growing tumor cells than normal cells. Several lines of evidence support the hypothesis that this increase in the quantity of protease released by the cancer cells contributes directly to the invasiveness of tumor cells and to the destruction of the adjacent host tissue. In the case of breast cancer metastases, four classes of proteases appear to be involved in disease progression. These four classes of proteases include cathepsins B and L (cysteine proteases), cathepsin D (aspartyl protease), collagenases (metallo-proteases) and urokinase and plasminogen (serine proteases). Proteases have been implicated in a number of malignant conditions and researchers have observed increased secretion of proteases into the interstitial fluid around growing tumors. These proteases inevitably act on proteins, including those in the coagulation cascade leading to the formation of fibrin. Furthermore fibrin is very frequently observed at the invading periphery of malignant neoplasms. Hiramoto et al. “Fibrin in Human Tumors,” Cancer Res., 20:592-593, (1960). Malignant cells also characteristically possess high levels of plasminogen activator which should induce local fibrinolysis. Ossowski, et al., “Fibrinolysis Associated with Oncogenic Transformation,” J. Exp. Med, 138:1056-1064, (1973).

The invasiveness of some tumor cells has been correlated with an increased expression of collagenase. Genetic manipulation of such tumor cells, in culture, to reduce the activity of the collagenase results in a decrease in the invasiveness of the cell and metastases caused by the cells, in vitro. Furthermore, the addition of tissue metalloproteinase inhibitors to tumor cells results in blocking of the cell's invasiveness in vitro. Similarly, the administration of either natural or synthetic metalloproteinase inhibitors prevents metastasis of lung cancer cells. One consequence of the release of proteases by tumor cells into the bloodstream is the proteolysis of serum proteins such as fibrinogen. Therefore, the extent of proteolytic degradation of serum proteins can be correlated with the activity of the tumor cells. Quantitatively the degree of proteolysis can be determined by measuring the quantity of the degradation products generated by the action of the proteases. This measurement is, therefore, an indirect estimate of the degree of malignancy of the tumor cells.

A significant feature of the invention is the identification of cancer markers which comprise epitopes on endogenous proteins that are generally inaccessible to immunodetection. Specifically, while these epitopes are usually masked by the factors such as the 3 dimensional structure of the protein, they become unmasked and accessible to immunodetection for example, upon proteolytic degradation that occurs in oncogenesis. With this knowledge, methods which measure unique epitopes that are either sterically or immunochemically unreactive in the native fibrinogen molecule and are manifested secondary to proteolytic degradation of fibrinogen are of particular interest. Further, in view of the concurrent increase in the formation of fibrin and in the secretion of proteases in malignant conditions, the measurement of serum fibrinogen degradation product (FDP) levels may represent a useful measure of malignancy. Specifically, methods to detect proteolytic degradation products of fibrinogen and other plasma proteins with minimal interference from the parent protein (the protease substrate) are of particular interest for use in a cancer detection assay. The results of studies establishing the viability of an immunoassay, called Oncochek, for the detection of FDPs as indicators of the presence of various cancers is described herein.

Within the present invention, peptides associated with oncogenic processes may be found in detectable concentrations in the biological samples of warm-blooded animals, including humans, possessing a disease which disrupts epithelial tissue. As disclosed in the present invention, unmasked peptides may be indicative of a variety of diseases and are detectable in a variety of samples, with or without purification of such peptides. For example, degradation peptides are shown to be associated with invasive cancers. Invasive cancers include cervical, urogenital (e.g., bladder and prostate), lung, colorectal, and head and neck cancers. Such peptides are also associated with epithelial disorders (i.e., non-invasive or pre-invasive cancers and disorders unrelated to cancer) including epithelial inflammations and collagen degenerative diseases.

Biological samples containing peptides associated with oncogenic processes may come a variety of sources. Representative types of biological samples include urine, cervical secretions, bronchial aspirates (including bronchial washings), sputum, saliva, feces, serum, synovial and cerebrospinal fluid. The type of biological sample in which peptides are present may depend chiefly on the location of the particular disease. For example, urine is preferred for the detection of invasive urogenital cancers and urogenital epithelial disorders. Cervical secretions are preferred for the detection of invasive cervical cancers and cervical epithelial disorders. Bronchial aspirates and sputum are preferred for the detection of invasive lung cancers and lung epithelial disorders. Knowledge of the site from which a bronchial aspirate is taken further permits one to identify the location of a disease within a lung. Saliva is preferred for head and neck cancers. Feces are preferred for invasive colorectal cancers and colorectal epithelial disorders. Cerebrospinal fluid is preferred for brain cancers. Alternatively, serum may be used for the detection of complexes as a “pan” marker (i.e., a general screening technique) from which follow-up tests would be recommended to identify the particular disease. It would be evident to those of ordinary skill in the art how to associate other biological samples with a particular disease location.

The presence or amount of a peptide may be determined in a variety of ways, including non-immunological and immunological. Non-immunological methodologies include the use of protein stains such as Coomassie blue or silver stains. In a preferred embodiment, a sample suspected of containing a peptide of interest is subjected to SDS-PAGE and identified using a protein stain. Other non-immunological methodologies include the use of radioisotopes and the like as reporter groups. Such methods are amenable to quantification where it is desired to determine the amount.

Alternatively, the presence or amount of a peptide associated with oncogenic processes may be detected by immunological means. Detection may be, for example, by Western blot analysis utilizing immobilized complexes or components thereof on nitrocellulose, or Immobilon or similar matrix in conjunction with specific antibodies to the peptides. Detection can also be achieved by immunoassay. In one embodiment, a peptide is isolated from a sample and contacted with an appropriate detection antibody. Complexes may be isolated by capture on a solid support (e.g., heparin agarose or polystyrene or heparin coated on polystyrene) or with a “capture” antibody prior to or simultaneous with a “detection” antibody. In another embodiment, peptide-antibody immunocomplexes are formed between an antibody and a peptide, without prior purification of the complex. Incubation of a sample with an antibody is under conditions and for a time sufficient to allow immunocomplexes to form. Detection of complexes or polypeptide constituents by immunological means is also amenable to quantification where it is desired to determine the amount of a peptide.

Detection of one or more immunocomplexes formed between a peptide and an antibody specific for the peptide may be accomplished by a variety of known techniques, including radioimmunoassays (RIA) and enzyme linked immunosorbent assays (ELISA). The immunoassays known in the art include the double monoclonal antibody sandwich immunoassay technique of David et al. (U.S. Pat. No. 4,376,110); monoclonal-polyclonal antibody sandwich assays (Wide et al., in Kirkham and Hunter (eds.), Radioimmunoassay Methods, E. and S. Livingstone, Edinburgh, 1970); the “western blot” method of Gordon et al. (U.S. Pat. No. 4,452,901); immunoprecipitation of labeled ligand (Brown et al., J. Biol. Chem. 255: 4980-4983, 1980); enzyme-linked immunosorbant assays as described by, for example, Raines and Ross (J. Biol. Chem. 257: 5154-5160, 1982); immunocytochemical techniques, including the use of fluorochromes (Brooks et al., Clin. Exp. Immunol. 39: 477, 1980); and neutralization of activity (Bowen-Pope et al., Proc. Natl. Acad. Sci. U.S.A. 81: 2396-2400, 1984), all of which are hereby incorporated by reference. In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; 4,098,876, and 5,591,595, all of which are herein incorporated by reference.

For detection purposes, the antibodies may either be labeled or unlabeled. When unlabeled, the antibodies find use in agglutination assays. In addition, unlabeled antibodies can be used in combination with other labeled antibodies (second antibodies) that are reactive with the antibody, such as antibodies specific for immunoglobulin. Alternatively, the antibodies can be directly labeled. Where they are labeled, the reporter group can include radioisotopes, fluorophores, enzymes, luminescers, or dye particles. These and other labels are well known in the art and are described, for example, in the following U.S. Pat. Nos. 3,766,162; 3,791,932; 3,817,837; 3,996,345; and 4,233,402.

Typically in an ELISA assay the target antigen or immobilized capture antibody is adsorbed to the surface of a microtiter well. Residual protein-binding sites on the surface are then blocked with an appropriate agent, such as bovine serum albumin (BSA), heat-inactivated normal goat serum (NGS), or BLOTTO (buffered solution of nonfat dry milk which also contains a preservative, salts, and an antifoaming agent). The well is then incubated with a sample suspected of containing specific antibody. The sample can be applied neat, or, more often, it can be diluted, usually in a buffered solution which contains a small amount (0.1%-5.0% by weight) of protein, such as BSA, NGS, or BLOTTO. After incubating for a sufficient length of time to allow specific binding to occur, the well is washed to remove unbound protein and then incubated with an anti-mouse immunoglobulin antibody labeled with a reporter group. The reporter group can be chosen from a variety of enzymes, including horseradish peroxidase, beta-galactosidase, alkaline phosphatase, and glucose oxidase. Sufficient time is allowed for specific binding to occur, the well is again washed to remove unbound conjugate, and the substrate for the enzyme is added. Color is allowed to develop and the optical density of the contents of the well is determined visually or instrumentally.

In one preferred embodiment of the present invention, a reporter group is bound to the antibody. The step of detecting an immuncomplex involves removing substantially any unbound antibody and then detecting the presence or absence of the reporter group.

In another preferred embodiment, a reporter group is bound to a second antibody capable of binding to the antibody specific for a peptide associated with an oncogenic process. The step of detecting an immunocomplex involves (a) removing substantially any unbound antibody, (b) adding the second antibody, (c) removing substantially any unbound second antibody, and then (d) detecting the presence or absence of the reporter group. Where the antibody specific for the fragment is derived from a mouse, the second antibody is an anti-murine antibody.

In another preferred embodiment for detecting an immunocomplex, a reporter group is bound to a molecule capable of binding to the immunocomplex. The step of detecting involves (a) adding the molecule, (b) removing substantially any unbound molecule, and then (c) detecting the presence or absence of the reporter group. An example of a molecule capable of binding to the immunocomplex is protein A.

It will be evident to one skilled in the art that a variety of methods for detecting the immunocomplex may be employed within the present invention. Reporter groups suitable for use in any of the methods include radioisotopes, fluorophores, enzymes, luminescers, and dye particles.

Taking advantage of the foregoing information, a method for detecting proteolytic degradation products of plasma proteins with minimal interference from the parent protein (the protease substrate) has been devised and used as a cancer detection assay. Specifically the method measures unique epitopes that are manifested secondary to proteolytic degradation of fibrinogen. These epitopes are either sterically or immunochemically unreactive in the native fibrinogen molecule. In addition to providing a general cancer assay, the invention provides a method for monitoring the course of a neoplastic condition by quantitatively determining the presence of peptides present in a biological sample over time.

The present invention is directed at a method for measuring the quantity of proteolytic degradation products of serum proteins. To overcome interference from undegraded, native serum proteins, a peptide contained within the interior of the native proteins is used. Such peptides are “masked” in the native protein and are not recognized or accessible by detection systems, such as antibodies, when the protein is intact. These “masked” peptides are not exposed until the protein is degraded and the internal peptides are released or exposed.

In one embodiment of the present invention, the method measures proteolytic degradation of fibrinogen with minimal interference from intact fibrinogen. In this embodiment of the present invention, two different antibodies are used as the detection system. One of the antibodies is specific for the peptide GHRPLDK which is part of the amino acid sequence of the β-chain of fibrinogen, located near its amino terminus.

Assay specificity is achieved by the use of two different antibodies in a two-site, solid-phase enzymometric assay. The more highly specific antibody, which is immobilized to the solid phase consists of a murine monoclonai to a glycine-histidine-arginine-proline-leucine-aspartate-lysine-cysteine (GHRPLDKC) octapeptide. The first seven amino acids of this peptide represent an internal sequence within the β-chain of fibrinogen, which is near the amino terminus and is exposed after initial plasminolysis (residues 15-21). Chung et al., “Characterization of Complementary Deoxyribonucleic Acid and Genomic Deoxyribonucleic Acid for the β Chain of Human Fibrinogen,” Biochemistry, 22:3244-3250, (1983). After capture of the proteolytic degradation products of fibrinogen by the immobilized monoclonal antibody, the immune complex is detected by using a highly specific conjugate consisting of polyclonal antifibrinogen antibody labeled with horseradish peroxidase.

While the peptide GHRPLDK has been used in one embodiment of the present invention, it will be clear to those skilled in the art that other internal fibrinogen peptides would also be of use, as would internal peptides of other proteins which are degraded by proteases produced by cancer. In an assay of the present invention a commercially available monoclonal antibody to the peptide GHRPLDKC can be used.

An illustrative antibody that is useful in this assay is the murine monoclonal antibody derived from clone DIG1OVL2 and which is commercially available from Biodesign International, Kennebunkport, Me. (Catalog number M42543M) and Immunotech, Inc., Westbrook, Me.). This monoclonal antibody was generated using an immunogen prepared from the peptide GHRPLDKC conjugated to bovine serum albumin. The sequence of the first 7 amino acids of the octapeptide corresponds to the amino acids number 15 to 21 of β-chain of human fibrinogen. The monoclonal antibody recognizes fragment D of fibrinogen but does not cross react with intact fibrinogen. In addition to recognizing fragment D, the monoclonal antibody also reacts with fibrinogen degradation products (FDP) produced by plasminolysis. However, the monoclonal antibody does not recognize fragment E. Fragment D is the proteolytic product of fibrinogen plasminolysis. Although, in the current assay format, the immobilized monoclonal antibody to fragment D will capture fragment D or FDP, only FDP are “sandwiched” by the polyclonal anti-fibrinogen antibody, labeled with horseradish peroxidase, which is used.

In use the monoclonal antibody was immobilized on a solid phase and used to capture proteolytic degradation products of fibrinogen. After being captured by the immobilized mouse monoclonal antibody, the degradation products were complexed by polyclonal antibody (ovine anti-human fibrinogen-peroxidase conjugate and which is commercially available from The Binding Site, Inc., San Diego, Calif.) to form an immuno-sandwich. While a sandwich enzyme linked immunosorbent assay (ELISA) was used in the Examples below, relating to this invention, one skilled in the art is aware that other assay formats can also be used.

As illustrated in the Examples below, assays for the peptides described above can be combined with tests for the presence of one or more known organ-specific tumor markers to increase the clinical sensitivity and enhance the diagnostic capacity of these assays. Such combination assays may be performed at the same time or sequentially. Those skilled in the art appreciate that there are a wide variety of known organ-specific tumor markers which are associated in varying degrees with different cancer lineages and which may be utilized in conjunction with the assays described herein (see e.g. Lamerz et al., “Serum Marker Combinations in Human Breast Cancer”, In Vivo 7(6B): 607-613 (1993). When used in conjunction with the recognized organ-specific tumor marker for breast, colon, and lung cancers the unique epitope detected by the Oncochek immunoassay system appears to offer increased clinical sensitivity.

The present invention is further detailed in the following Examples, which are offered by way of illustration and are not intended to limit the invention in any manner. Standard techniques well known in the art or the techniques specifically described below are utilized. All patent and literature references cited in the present specification are hereby incorporated by reference in their entirety.

### EXAMPLES

Commercially available reagents referred to in the examples were used according to manufacturer's instructions unless otherwise indicated.

### Example 1

Coating of Antibody onto 96-Well Microtiter Plates

The monoclonal anti-fibrinogen-peptide antibody (Clone D1G1OVL2) was dissolved and diluted to 2 μg/ml in pH 8.8 borate buffer (0.125 M Borate, pH 8.8, 0.225 M NaCl, 5 mM EDTA, 50 mM 3-amino-m-caproic acid, 10 μg/ml 4-aminobenzamidine-HCl). 120-μl aliquots of the diluted antibody solution were added to each well of each microtiter plates (96-well microtiter plates obtained from Fisher Scientific, Fair Lawn, N.J.) and incubated overnight (15-20 hr) at 25° C.

The microtiter plates where then washed twice with Tris buffered saline, pH 7.4 (TBS: 2.5 mM Tris, pH 7.4, 13.7 mM NaCl, 0.3 mM KCl, 0.002% (v/v) TWEEN-20, 0.001% (v/v) Triton X-100, 5 μg/ml gentamicin, 2.5 μg/ml amphotericin B). 300-μl of STABILCOAT™ (obtained from BSI Corp., Eden Prairie, Minn.) was added to each well of each microtiter plate, and the plates were incubated at 25° C. for at least 2 hours. The STABILCOAT™ was then removed from the wells of the microtiter plates and the plates were dried overnight in a vacuum desiccator.

### Example 2

Preparation of Plasmin-Digested Fibrinogen (FDP)

For Used as Calibrators

Fibrinogen was plasmin-digested according to the method of Haverkate and Timan as setforth below.

Fibrinogen (obtained from Sigma Chemical Co., St. Louis, Mo.) was dissolved at a concentration of 0.15% (w/v) in 0.05 M MOPS, pH 7.4, 0.10 M NaCl and 2 mM CaCl₂. Plasmin (obtained from Sigma Chemical Co., St. Louis, Mo.) was added to a final concentration of 0.25 units per ml fibrinogen solution, and the mixture was incubated at 37° C. for 3 hours. At the end of the 3 hour incubation, the FDP was frozen until required.

For use as calibrators the FDP sample was diluted with phosphate buffered saline (PBS: 137 mM NaCl, 1.6 mM KCl, 8.1 mM Na₂HPO₄, 1.5 mM KH₂PO₄) with 5 mM EDTA and 1% (w/v) BSA.

### Example 3

Assay Procedure

All calibrators, controls, and samples were diluted 1:200 with diluent buffer (PBS with 5 mM EDTA and 1% (w/v) BSA). 100-μl aliquots of diluted calibrators, controls, or samples were added to the wells of coated microtiter plates (coated as described in Example 1) and incubated for 30 minutes at 25° C. At the end of the incubation, the microtiter plates were washed six times with TBS. Then 100 μl of antibody-peroxidase conjugate solution was added to the wells of the microtiter plate and the plates were incubated for 30 minutes at 25° C. At the end of the incubation the microtiter plates were washed six times with TBS. 100-μl of TMB (substrate for the horseradish peroxidase obtained from Kirkegaard & Perry Laboratories, Inc., Gaithersburg, Md.) was then added to each well, and the plates were incubated for 15 minutes at 25° C. At the end of the incubation 100 μl stop solution (0.1 M HCl) was added to each well. The solution in the wells of the microtiter plates was then read at 450 nin.

### Example 4

Statistical Analysis

The assay sensitivity-specificity relationship was analyzed using ROC (receiver-operating characteristic) plots that were constructed by measuring the levels of FDP from sera of both cancer patients and normal control subjects. Such an analysis is a powerful means to describe diagnostic accuracy of the assay. The diagnostic sensitivity is defined by equation 1:
Sensitivity=True Positives/(True Positives+False Negatives)
and the diagnostic specificity is defined by equation 2:
Specificity=True Negatives/(True Negative+False Positives)

The comparative ability of fragment D, fragment E, intact fibrinogen and FDP to form sandwiches between the monoclonal and polyclonal antibodies are summarized in Table I.

### Example 5

Calibration Curve

The calibrators for the assay were prepared by plasminolysis of fibrinogen as described in Example 2. Intact fibrinogen (fibrinogen not subjected to prior treatment with plasmin) was unreactive in the assay of the present invention whereas immunoreactive FDP were formed from fibrinogen by plasmin treatment in a time-dependent fashion (Table I).

TABLE I
Analytical Specificity of the Assay

| | Fibrinogen | Fibrinogen | | |
|---|---|---|---|---|
| Sample tested | Fragment D | Fragment E | Fibrinogen | FDP |
| Concentration μg/ml | 100 | 100 | 100 | 100 |
| Absorbance at 450 | 0.045 | 0.064 | 0.088 | 1.500 |
| nm | | | | |

The results in Table I show that neither fibrinogen fragment D, fibrinogen fragment E nor intact fibrinogen show significant reaction in the assay of the present invention. However, the fibrinogen digested with plasmin results in significant immuno-reaction in the assay of the present invention.

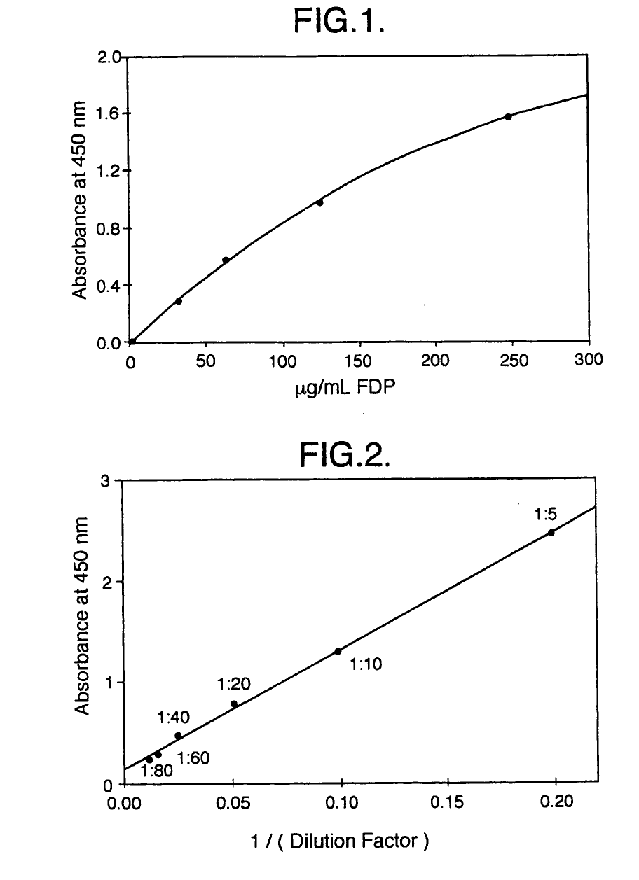

FIG. 1 shows a standard curve for the reaction of different concentrations of FDP (over the range of 32 to 250 μg/ml) with the assay system of the present invention. The results indicate that the absorbance at 450 nm is proportional to the amount of FDP added, over the range studied. The immunoreactive products present in the serum of a cancer patient with high levels of FDP exhibited linearity in dilutional parallelism to the FDP calibration curve over a dilution range from 5- to 80-fold (see FIG. 2).

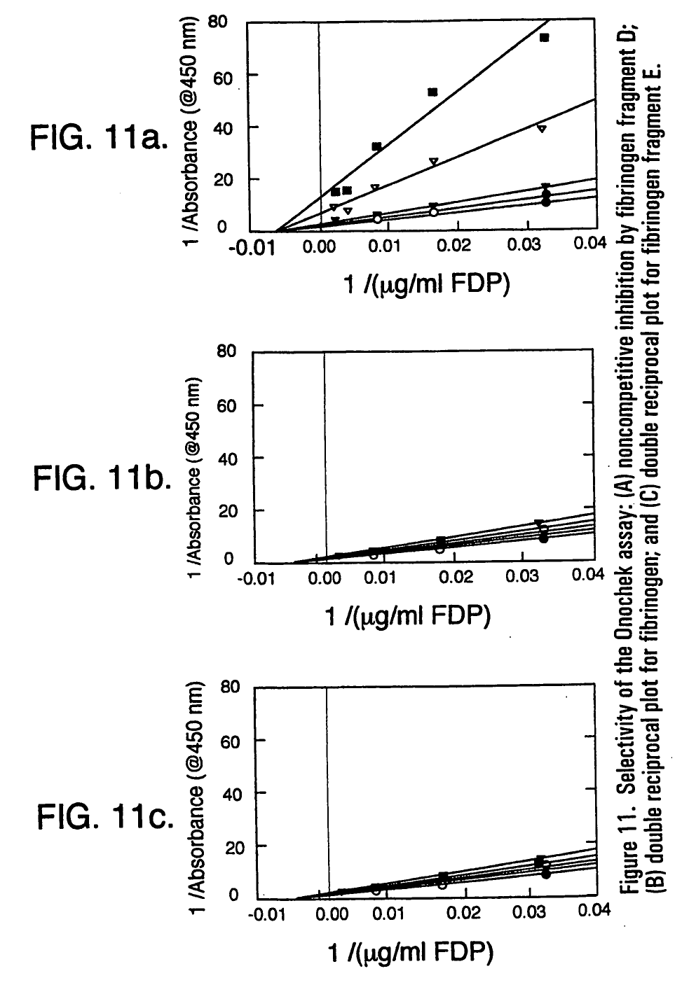

FIG. 11(A) illustrates results indicating that FD affects FDP measurements in the Oncochek assay in a pattern consistent with noncompetitive inhibition or covert cross-reactivity. Suelter C H., A. Practical Guide to Enzymology, New York, Wiley, p. 248, (1985). This inhibition pattern is consistent with the mechanism that FD binds to the solid phase of capture antibody, thus reducing the antibody sites available for binding FDPs. The double reciprocal plots of FE and FG inhibition studies are consistent with the absence of interaction between MAb and FE and FG (see FIGS. 11(B) and 11(C)). They are also consistent with the results presented in Table I, which shows the lack of response by FE and FG in the Oncochek assay.

### Example 6

Studies Using Clinical Samples

Sera from fifty control patients (non-cancer) and sixty-five cancer patients were obtained from Orange Coast Hematology and Oncology Groups, Poland Institute of Oncology, Austin Medical Ventures, and LA Metropolitan Hospital. The segmentation of the cancer patient group included 12 lung cancer patients, 10 breast cancer patients, 11 prostate cancer patients, 18 ovarian cancer patients, and 14 colon cancer patients.

Table II shows the FDP levels in the sera of the 50 normal (Table IIa) control subjects and the 65 cancer patients (Table IIb).

TABLE II(a)
Measurement Of The FDP Levels In
Control Subjects (Non-Cancer)

| | | Dₘ/F¹ Assay |
|---|---|---|
| Sample # | Gender | (μg/ml FDP) |
| 1 | M | 33 |
| 2 | F | 19 |
| 3 | F | 86 |
| 4 | F | 27 |
| 5 | F | 91 |
| 6 | F | 35 |
| 7 | F | 14 |
| 8 | F | 12 |
| 9 | F | 26 |
| 10 | F | 69 |
| 11 | F | 0 |
| 12 | F | 30 |
| 13 | F | 3 |
| 14 | F | 0 |
| 15 | F | 0 |
| 16 | F | 96 |
| 17 | F | 0 |
| 18 | F | 0 |
| 19 | F | 0 |
| 20 | M | 0 |
| 21 | M | 0 |
| 22 | F | 72 |
| 23 | F | 129 |
| 24 | F | 51 |
| 25 | F | 0 |
| 26 | F | 215 |
| 27 | F | 50 |
| 28 | F | 207 |
| 29 | F | 0 |
| 30 | F | 133 |
| 31 | F | 59 |
| 32 | U² | 57 |
| 33 | F | 96 |
| 34 | M | 0 |
| 35 | M | 122 |
| 36 | M | 102 |
| 37 | F | 160 |
| 38 | M | 103 |
| 39 | M | 0 |
| 40 | M | 99 |
| 41 | M | 71 |
| 42 | M | 0 |
| 43 | F | 84 |
| 44 | F | 148 |
| 45 | F | 111 |
| 46 | F | 88 |
| 47 | M | 7 |
| 48 | M | 37 |
| 49 | M | 0 |
| 50 | F | 0 |

¹Dm/F = ELISA using monoclonal anti-fibrinogen-peptide antibody and polyclonal anti-fibrinogen conjugated to horse radish peroxides.
²U = Unknown

TABLE IIb
FDP Level In Sera Of Cancer Patients

| | | |
|---|---|---|
| | | Dₘ/F Assay |
| Sample # | Gender | (μg/ml FDP) |

Lung Cancer Patents

| | | |
|---|---|---|
| 1 | M | 132 |
| 2 | F | 161 |
| 3 | M | 456 |
| 4 | F | 0 |
| 5 | F | 26 |
| 6 | M | 106 |
| 7 | F | 311 |
| 8 | F | 300 |
| 9 | M | 377 |
| 10 | M | 15 |
| 11 | F | 0 |
| 12 | M | 0 |

Breast Cancer Patients

| | | |
|---|---|---|
| 1 | F | 14 |
| 2 | F | 0 |
| 3 | F | 0 |
| 4 | F | 81 |
| 5 | F | 215 |
| 6 | F | 0 |
| 7 | F | 101 |
| 8 | F | 0 |
| 9 | F | 0 |
| 10 | F | 0 |

Prostate Cancer Patients

| 1 | M | 113 |
|---|---|---|
| 2 | M | 192 |
| 3 | M | 0 |
| 4 | M | 345 |
| 5 | M | 17 |
| 6 | M | 251 |
| 7 | M | 371 |
| 8 | M | 129 |
| 9 | M | 167 |
| 10 | M | 270 |
| 11 | M | 451 |
| | | Dₘ/F¹ Assay |
| Sample # | Gender | (μg/ml FDP) |

Ovarian Cancer Patients

| | | |
|---|---|---|
| 1 | F | 33 |
| 2 | F | 252 |
| 3 | F | 37 |
| 4 | F | 8 |
| 5 | F | 215 |
| 6 | F | 0 |
| 7 | F | 23 |
| 8 | F | 196 |
| 9 | F | 107 |
| 10 | F | 108 |
| 11 | F | 165 |
| 12 | F | 371 |
| 13 | F | 125 |
| 14 | F | 167 |
| 15 | F | 195 |
| 16 | F | 162 |
| 17 | F | 154 |
| 18 | F | 144 |

Colon Cancer Patients

| | | |
|---|---|---|
| 1 | F | 0 |
| 2 | M | 0 |
| 3 | M | 510 |
| 4 | F | 9 |
| 5 | M | 134 |
| 6 | M | 0 |
| 7 | F | 211 |
| 8 | F | 222 |
| 9 | M | 80 |
| 10 | M | 236 |
| 11 | M | 17 |
| 12 | M | 0 |
| 13 | F | 47 |
| 14 | M | 52 |

Samples from cancer patients generally exhibited higher concentrations of FDP, using the Dₘ/F assay format, than did control patients.

Based on the data presented in Table II, an ROC analysis of the assay was performed to obtain information on the relationship between the sensitivity and specificity of the assay. The result of the ROC analysis is presented in Table III which indicates that, using an FDP level of 150 μg/ml, the specificity of the assay is 94% and the sensitivity was 42%, 64% and 50% for lung, prostate and ovarian cancer, respectively. The assay was shown to be highly specific for FDP and little or no cross-reaction was observed with fibrinogen fragment D, fibrinogen fragment E, or intact fibrinogen.

TABLE III
ROC Analysis of Sensitivity and Specificity

| | | Specificity | Sensitivity | | | | | |
|---|---|---|---|---|---|---|---|---|
| | | Normal | All Cancer | | | | | |
| FDP Cutoff | | Sera | Patients | Lung | Breast | Prostate | Ovarian | Colon |
| Level (μg/ml) | | n = 50 | n = 65 | n = 12 | n = 10 | n = 11 | n = 18 | n = 14 |
| 75 | fr¹ | 33/50 | 38/65 | 7/12 | 3/10 | 9/11 | 13/18 | 6/14 |
| | % | 66% | 58% | 58% | 30% | 82% | 72% | 43% |
| 90 | fr | 36/50 | 36/65 | 7/12 | 2/10 | 9/11 | 13/18 | 5/14 |
| | % | 72% | 55% | 58% | 20% | 82% | 72% | 36% |
| 105 | fr | 42/50 | 35/65 | 7/12 | 1/10 | 9/11 | 13/18 | 5/14 |
| | % | 84% | 54% | 58% | 10% | 82% | 72% | 36% |
| 120 | fr | 43/50 | 34/65 | 6/12 | 1/10 | 8/11 | 11/18 | 5/14 |
| | % | 86% | 52% | 50% | 10% | 73% | 61% | 36% |
| 130 | fr | 45/50 | 29/65 | 6/12 | 1/10 | 7/11 | 10/18 | 5/14 |
| | % | 90% | 45% | 50% | 10% | 64% | 56% | 36% |
| 135 | fr | 46/50 | 27/65 | 5/12 | 1/10 | 7/11 | 10/18 | 4/14 |
| | % | 92% | 42% | 42% | 10% | 64% | 56% | 29% |
| 150 | fr | 47/50 | 26/65 | 5/12 | 1/10 | 7/11 | 9/18 | 4/14 |
| | % | 94% | 40% | 42% | 10% | 64% | 50% | 29% |

¹fr = fraction

The results shown in Tables II and III demonstrated that the assay of the present invention is capable of detecting more than one type of cancer with a high degree of specificity and an acceptable degree of sensitivity.

### Example 7

Clinical Performance of FDP Relative to Other Markers

Sera from control patients (non-cancer) and from patients with breast, colon, lung, ovarian or prostate cancer were obtained from a commercial supplier. Fifty samples were used in each group.

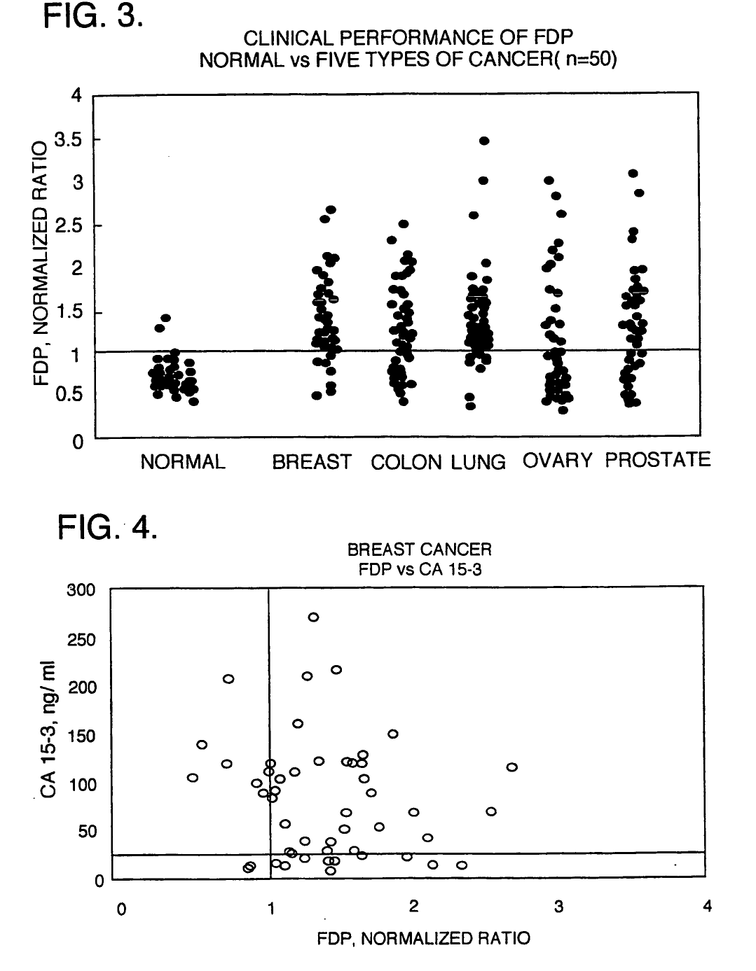

FDP levels were measured and normalized such that a normalized ratio of 1.0 represents the upper limit of the normal range. FIG. 3 shows the results of these measurements for each group.

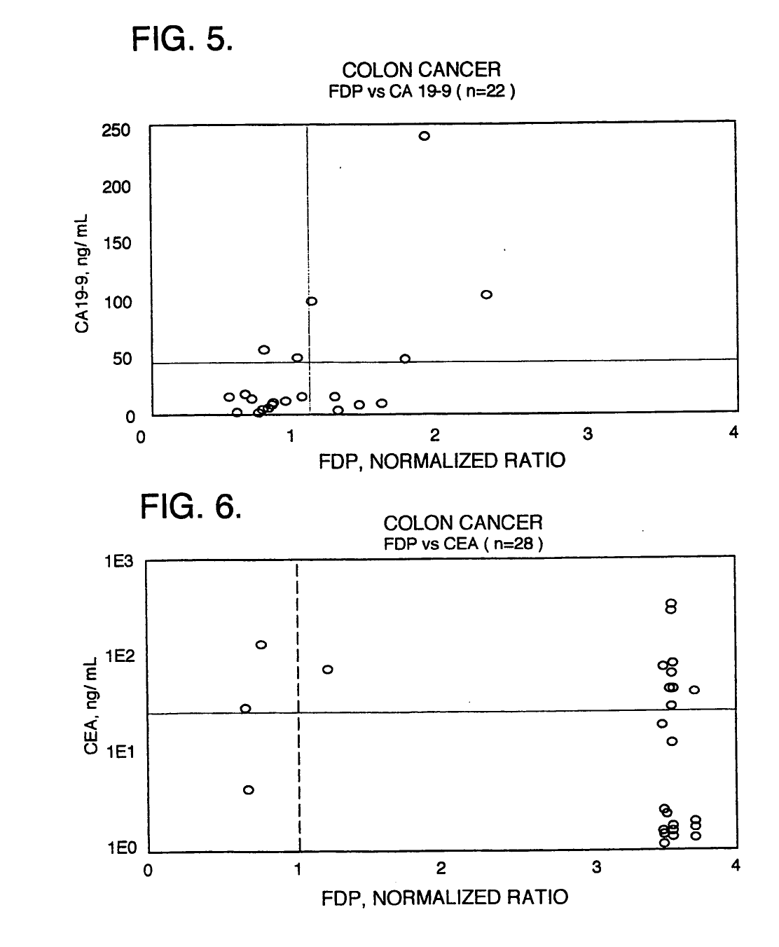

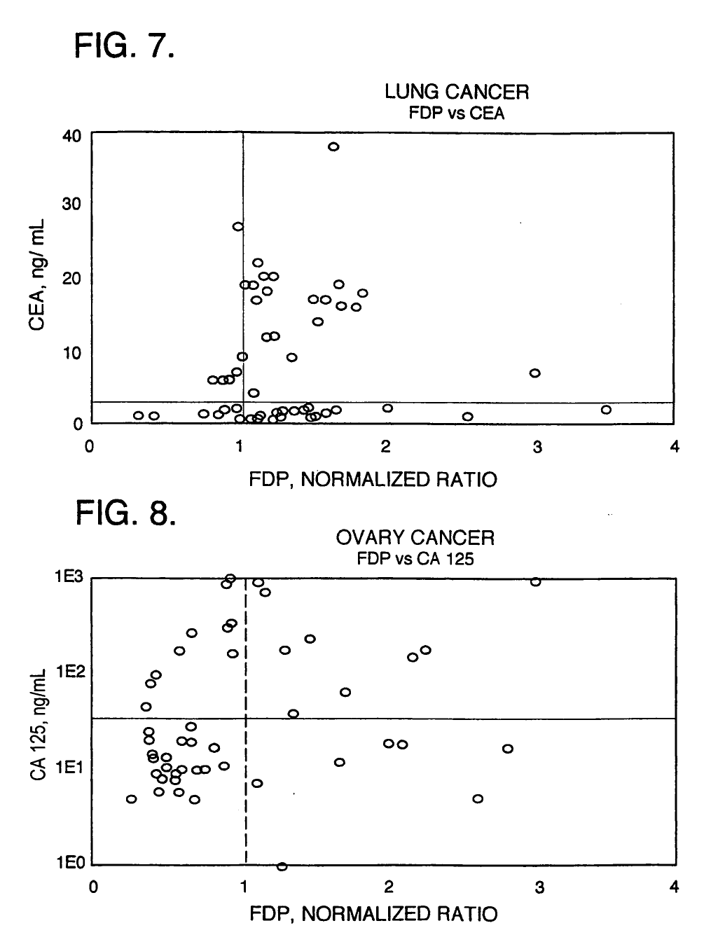

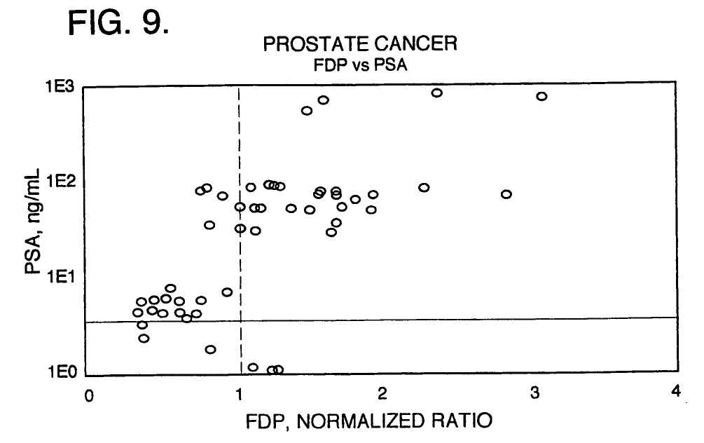

Levels of known cancer antigens were also measured in the same samples and these levels were compared to the normalized ratios of FDP. FIG. 4 is a scatterplot of CA 15-3 levels as compared to FDP normalized ratio for individual samples from breast cancer patients. FIG. 5 is a scatterplot of CA19-9 levels as compared to FDP normalized ratio for 22 of the 50 individual samples from colon cancer patients. FIG. 6 is a scatterplot of CEA levels as compared to FDP normalized ratio for 28 of the 50 individual samples from colon cancer patients. FIG. 7 is a scatterplot of CEA levels as compared to FDP normalized ratio for individual samples from lung cancer patients. FIG. 8 is a scatterplot of CA 125 levels as compared to FDP normalized ratio for individual samples from ovarian cancer patients. FIG. 9 is a scatterplot of PSA levels as compared to FDP normalized ratio for individual samples from prostate cancer patients.

These scatterplots demonstrate the increased sensitivity of FDP measurements relative to measurement of other cancer antigens. This increased sensitivity is particularly demonstrated by the datapoints which fall within the lower right quadrant of the plots. The results presented in FIG. 3 show that FDP measurements detect a wide variety of cancers.

Results of the Oncochek assay indicate that FDP levels in the sera of patients with various types of cancer are significantly elevated in comparison to normals. For example, FDP levels in the sera of normal control subjects were compared with those in the sera of patients with five types of cancers. Each group consisted of 50 patients and included breast, colon, lung, ovarian, and prostate cancers. The data presented in FIG. 3 were subjected to a receiver-operating-characteristics (ROC) analysis to assess the relationship between the sensitivity and specificity of the assay at various threshold concentrations of FDP.

By ROC analysis using an upper limit of normal corresponding to 96% specificity sensitivities of 84, 82, 82, 34, and 60% were achieved for breast, colon, lung, ovarian, and prostate cancers, respectively (see Table IV below). If an elevation in the value of either the Oncochek assay or the organ-specific marker (or both) was used as a prediction of the presence of cancer, sensitivities approximating 90% or greater were achieved for breast, colon, and lung cancers.

TABLE IV

| | | | Observed Sensitivity (%) | | |
|---|---|---|---|---|---|
| Organ | Marker | N= | Oncochek | Marker | Both |
| Breast | CA 15-3 | 50 | 84 | 62 | 96 |
| Colon | CA 19-9 | 22 | 36 | 27 | 45 |
| | CEA | 28 | 82 | 50 | 89 |
| Lung | CEA | 50 | 82 | 52 | 90 |
| Ovary | CA 125 | 50 | 34 | 42 | 56 |
| Prostate | PSA | 50 | 60 | 84 | 90 |

Results shown in Table IV and FIG. 3 suggest that the Oncochek immuno-assay can detect multiple cancers with a high degree of specificity and clinical sensitivity. When it is used with established organ-specific markers, improved clinical sensitivity may be achieved for breast, colon, and lung cancers.

### Example 8

Improved Specificity of FDP Relative to Other Markers

Samples of pleural effusate from a lung cancer patient were prepared for sodium dodecylsulfate polyacrylamide electrophoresis (SDS-PAGE) gels and transferred to nitrocellulose for western blotting using standard methods well known in the art.

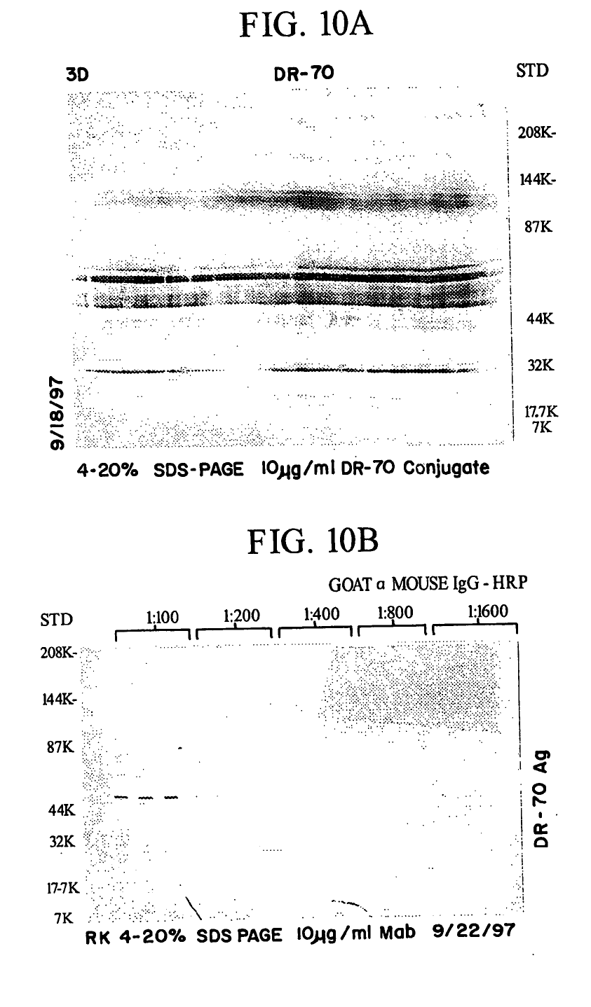

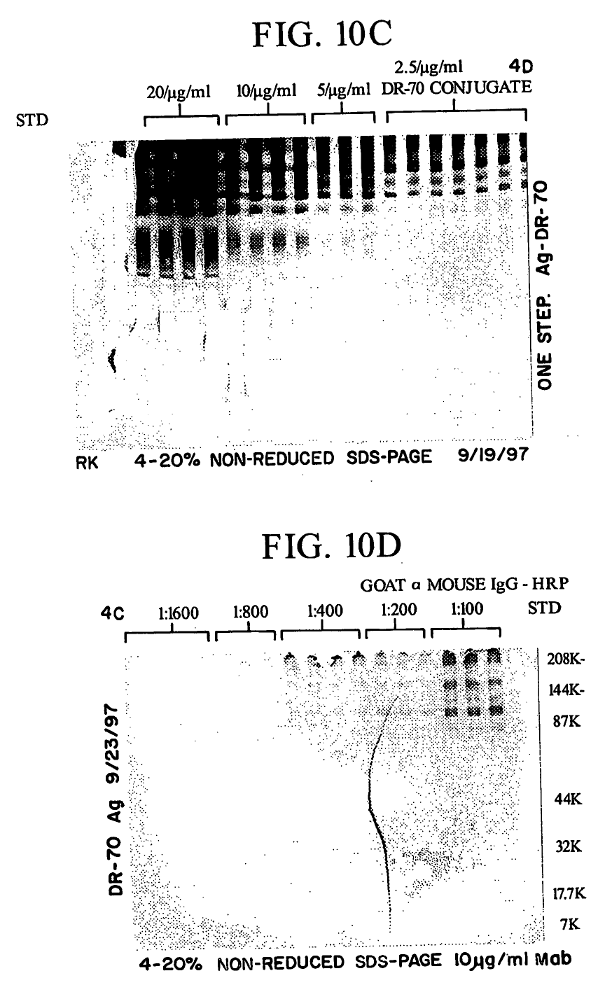

FIGS. 10a-d show results from reduced (with mercaptoethanol; FIGS. 10a-b) and non-reduced (without mercaptoethanol; FIGS. 10c-d) gels. In FIGS. 10a and 10c, the probe was derived from a ring shaped particle extract. (Ring shaped particles are described in U.S. Pat. No. 5,635,605, issued Jun. 3, 1997, and U.S. Pat. No. 5,459,035, issued Oct. 17, 1995.) In FIGS. 10b and 10d, the probe was the monoclonal antibodies of the invention.

These results show that the molecules of the invention can be used to detect cancer with a much higher specificity than obtained with other cancer detection probes.

The above description is of one embodiment of the present invention. However, it will be clear to those skilled in the art that various changes and modifications may be made without departing from the spirit of the invention.

What is claimed is:

1. A method for detecting cancer in a subject comprising contacting a biological sample obtained from the subject with an antibody that binds an epitope on a blood protein degradation peptide that is masked in the blood protein and determining the presence of an antibody-peptide complex.

2. The method of claim 1, wherein the blood protein is fibrinogen.

3. The method of claim 2, wherein the antibody recognizes an epitope comprising the amino acids 15 to 21 of the β-chain of human fibrinogen.

4. The method of claim 3, wherein the antibody is a monoclonal antibody.

5. The method of claim 1, wherein the presence of the antibody-peptide complex is determined by an assay comprising an enzyme-linked immunoadsorbent assay.

6. The method of claim 1, further comprising the step of screening a biological sample isolated from the subject for the presence of a second tumor marker.

7. The method of claim 6, wherein the second tumor marker is selected from the group consisting of PSA, CEA, CA 15-3, CA 19-9 and CA 125.

8. The method of claim 1, wherein the subject is a mammal.

9. The method of claim 8, wherein the subject is a human.

10. The method of claim 1, wherein the biological sample is a blood sample.

11. A method of detecting the presence of a fibrinogen degradation peptide associated with cancer in a biological sample comprising contacting the biological sample with an antibody that binds the degradation peptide and determining the presence of an antibody-peptide complex.

12. The method of claim 11, wherein the antibody recognizes an epitope comprising the amino acids 15 to 21 of the β-chain of human fibrinogen.

13. The method of claim 12, wherein the antibody is a monoclonal antibody.

14. The method of claim 11, wherein the presence of the antibody-peptide complex is determined by an assay comprising an enzyme-linked immunoadsorbent assay.

15. The method of claim 14, wherein the antibody is immobilized to a solid support.

16. The method of claim 15, wherein the enzyme-linked immunoadsorbent assay comprises a capture immunoassay wherein the antibody-peptide complex is detected with a second antibody which binds the peptide.

17. The method of claim 16, wherein the second antibody is joined to a detectable label.

18. The antibody of claim 17, wherein the detectable label is selected from the group consisting of radioactive isotopes, enzymes, or chromophores.

19. A method of detecting a disease process associated with the degradation of fibrinogen in a mammal comprising testing a biological sample isolated from the mammal for the presence of a peptide having an unmasked fibrinogen epitope by contacting the blood sample isolated from the mammal with an antibody specific for the peptide and determining the presence of an antibody-peptide complex.

20. The method of claim 19, wherein the antibody recognizes an epitope comprising the amino acids 15 to 21 of the β-chain of human fibrinogen.

21. The method of claim 20, wherein the antibody is a monoclonal antibody.
